(19) United States
(12) Patent Application Publication
(10) Pub. No.: US 2025/0115961 A1
(43) Pub. Date: Apr. 10, 2025

(54) DIAGNOSIS AND TREATMENT OF MÉNIÈRE'S DISEASE (MD)

(71) Applicant: The Regents of the University of California, Oakland, CA (US)

(72) Inventors: Rick A. FRIEDMAN, La Jolla, CA (US); Jeffrey P. HARRIS, La Jolla, CA (US); Kathleen FISCH, La Jolla, CA (US); Kristen JEPSEN, La Jolla, CA (US)

(73) Assignee: The Regents of the University of California, Oakland, CA (US)

(21) Appl. No.: 18/729,605

(22) PCT Filed: Jan. 19, 2023
(86) PCT No.: PCT/US2023/060891

§ 371 Date: Jul. 17, 2024

Related U.S. Application Data
(60) Provisional application No. 63/301,738, filed on Jan. 21, 2022.

(51) Int. Cl. C12Q 1/6883

(52) U.S. Cl. CPC C12Q 1/6883; C12Q 2600/156

(57) ABSTRACT

A method of diagnosis of Ménière's disease (MD) by performing genetic testing using a panel of gene mutations, including single nucleotide polymorphisms (SNPs), insertions and/or deletions (IN/DELs), in Ménière's disease-associated genes, providing genetic basis for MD. A method of treatment of MD by targeting the SNPs and/or IN/DELs gene mutations in Ménière's disease-associated genes, and/or by CRISPR or gene therapy or editing on the SNPs and/or IN/DELs gene mutations in Ménière's disease-associated genes.

### CROSS-REFERENCE TO RELATED APPLICATION

This application claims the benefit of U.S. Provisional Application No. 63/301,738, filed on Jan. 21, 2022, the entire content of which is incorporated herein by reference.

### STATEMENT REGARDING FEDERALLY SPONSORED RESEARCH OR DEVELOPMENT

This invention was made with government support under TR001442 awarded by the National Institutes of Health. The government has certain rights in the invention.

### BACKGROUND OF THE INVENTION

Ménière's disease (MD) is a disorder of the inner ear that can lead to severe dizziness, roaring tinnitus, and fluctuating hearing loss, usually starts in one ear, but later may involve both. Smoking, infections, or a high-salt diet may worsen the disease. Symptoms include a spinning sensation (vertigo), hearing loss, ear ringing (tinnitus), and ear pressure. The vertigo may cause severe nausea and imbalance. Hearing loss may become permanent. The disease affects approximately 12 out of every 1,000 people, is most often sporadic but can occur in a familial form in roughly 5% of cases.

Ménière's disease (MD) (MD) was first described by Prosper Meniere, in 1861. Although the cause is unknown, human temporal bone studies have linked MD symptoms to elevated pressure within the inner ear—specifically, the endolymphatic cochlear compartment (scala media). It is believed that this endolymphatic hydrops begins with derangement of the ionic composition of the scala media. The symptoms of the disease—tinnitus, vertigo, and hearing loss—are managed with salt restriction, diuretics, vestibular suppressants, and corticosteroids. Nonetheless, 60 percent of patients progress to severe hearing loss and persistent disequilibrium. To date, the true etiology of the disease remains unknown.

The NIDCD estimates that there are 615,000 Americans with MD and the disease accounts for 45,500 patient visits each year. Although very little literature exists on the socioeconomic impact of MD, a study from Sweden followed 19 patients over a 3-year period to assess the impact on productivity (Cohen & Jenkin, 1995, PMID: 7803019). It was concluded that the costs to society and the patients were substantial with 1,536 days of sick leave requested by these 19 subjects. In addition to these lost days, there is the tremendous cost of surgery, lost productivity due to agoraphobia and the impact of drop attacks on vocation, driving, and the activities of daily living (Lopez-Escamez et al., 2009, PMID: 19663372). Taken together, these data suggest that patients with this disease are in dire need of therapeutics and that the cost to benefit ratio for therapeutic development is small.

To date, little is known about the underpinnings of Ménière's disease (MD). The pathophysiology is somewhat elusive; however, most clinicians believe it is the result of altered labyrinthine fluid mechanics resulting in endolymphatic hydrops. Several lines of evidence support a genetic basis for MD. First and perhaps most conclusively, familial cases are not uncommon. A number of studies have suggested a familial incidence for MD of between 5% and 20% (Morris et al., 1994, PMID: 8109627; Morris et al., 2009 PMID: 18616841; Jen, 2011 PMID: 21358195} with one linkage study in a large Scandinavian family identifying a region on human chromosome 12p12.3 linked to MD (Klar et al., 2006 PMID: 16741942; Gabrikova et al., 2010 PMID: 20927121). A second line of evidence for a genetic predisposition to MD comes from candidate gene studies. Both immune based (PTPN22, HCF1 and HLA-DRB) and non-immune based (KCN1, KCN3, ADD1, AQP4 and COCH) genes may be associated with MD (Teggi et al., 2008 PMID: 18667944; Maekawa et al., 2010 PMID: 20722976; Candreia et al., 2010 PMID: 21063116}. An initial genome-wide analysis of patients with MD disease was published and it was demonstrated a clear racial predilection (Caucasians) supporting the notion of a genetic etiology. The clear predilection of the disease for Caucasians over other ethnicities supports a genetic component. A number of reports have documented disease frequencies in different ethnicities and have shown that Caucasians are more susceptible than Asians, who in turn are more susceptible than people of African ancestry (Ohmen et al., 2013 PMID: 23598705).

An autoimmune basis for MD has always been strongly considered. A subgroup analysis of patients with definite MD found alleles (rs3774937 and rs4648011) of NF-KB that were associated with faster progression of hearing loss (Cabrera et al., PLOS One 9, e112171, 2014). Polymorphisms in IL-1 and MIF were associated with MD in Japanese and European cohorts, respectively (Furuta et al., Int. J. Immunogenet. 38, 249-254, 2011; Gázquez et al., Eur. Arch. Otorhinolaryngol. 270, 1521-1529, 2013). However, up to now, no replication of these studies has been published.

Investigations into ion channelopathies have led to some indication of an association but as yet have not been replicated. For example, polymorphisms in KCNE1 and 3 were identified in a Japanese population (Doi et al., KCNE1 and KCNE3. ORL J. Otorhinolaryngol. Relat. Spec. 67, 289-293, 2005). However, subsequent replication and a meta-analysis failed to replicate these results (Campbell et al., Am. J. Med. Genet. A 152A, 67-74, 2010; Hietikko et al., Int. J. Audiol. 51, 841-845, 2012); Li, et al., J. Vestib. Res. 25, 211-218, 2016). Furthermore, abnormal water channels of the inner ear have been implicated in the development of endolymphatic hydrops. The genetic variants rs426496, rs591810, and rs3736309 in AQP 2, AQP3, and AP5, respectively, were identified in case control studies among patients in Brazil94 and Japan95 indicating a genetic basis for AQP-mediated theories of the of MD (Lopes et al., Otol. Neurotol. 37, 1117-1121, 2016; Nishio et al., Life Sci. 92, 541-546, 2013).

To date, no targeted therapy exists. Drugs for motion sickness or nausea may help manage symptoms. Treatment consists of anti-nausea medications. Drugs for motion sickness or nausea may help manage symptoms. Diuretic, antiemetic, antihistamine and/or sedative are also medications used for managing symptoms.

### SUMMARY OF THE INVENTION

The present disclosure provides the largest whole-genome sequencing study on carefully diagnosed unilateral MD patients with the goal of gene/pathway discovery and targeted intervention. More specifically, the present disclosure provides a method of diagnosis of a patient with Ménière's disease (MD) by performing genetic testing using a panel of Ménière's disease-associated single nucleotide polymorphisms (SNPs), insertions and deletions (IN/DELs). The genes in the diagnostic panel disclosed in the present disclosure includes, but is not limited to, SNPs and/or IN/DELs in genes, such as FAM136A, ANKRD36, ANKRD36C, RBFOX1, GXYLT1, FAM8A1, ATP2B2,AP3S1, DYNC1H1, FLNA, KIFAP3 and AARS1.

The present disclosure further provides a method of treating Ménière's disease (MD) by targeting the pathways disrupted by the discovered SNPs and/orIN/DELs disclosed herein via administering a drug, such as Amiloride, and/or other pyrazinoylguanidine class of compounds, to a patient in need. Intratympanic administration is a prime method of administration, but other methods can also be used.

The disclosure further provides a method of treating Ménière's disease (MD) using CRISPR or gene therapy or editing on the SNPs and/orIN/DELs in certain genes disclosed herein.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the drawings with the descriptions. The components in the drawings and descriptions are not necessarily to scale, emphasis instead being placed upon clearly illustrating the principles of the present disclosure. Moreover, in the drawings, like reference numerals designate corresponding parts throughout the several views.

FIG. 1. 281 variants recurrently mutated in 10 or more samples were observed. These include an in-frame deletion in RBFOX1 (p.Ser31del), found mutated in 258 patients (˜50%), an 18 fold increase over expectation, a missense mutation in FAM136A (p.Asp195His), mutated in 153 samples, a 9-fold increase over expectation, a nonsense mutation in GXYLT1 (p.Leu223_Arg224delinsTer), found in 151 samples, an 87 fold-increase over expectation, and a missense mutation in FAM8A1 (p.Leu135Pro), found in 99 samples, a 16-fold increase over expectation. One of the most striking variants in these data is a frameshift deletion in ATP2B2, a known variant (p.Thr927Ter; rs1438373024) so rare that it is not observed a single time in the gnomad V3 database, but is observed 48 times in these data.

FIG. 2. Integrated all genes mutated in more than 10 patients with a public database connecting mouse genotypes to phenotypes. There are 816 such genes in these data. A significant enrichment was found between these 816 genes and phenotypes related to abnormal hearing, auditory brainstem response, abnormal cochlear morphology, and impaired balance. Such an overlap between genes found recurrently mutated in MD and those same genes impacting relevant phenotypes in mouse when mutated provides evidence that genes and variants identified herein drive MD.

FIG. 3. By leveraging prior biological knowledge in the form of molecular interaction networks, biological pathways, and network propagation, the list of genes and variants in the MD cohort was reprioritized. The reprioritized list boosts signal from real variants, while downweighting false positives, since false positive hits are less likely to interact in the network. 77 genes were identified with z>7. Of these reprioritized genes, ATP2B2, ACAT2, AK2, PC, and MAT2A have a variant mutated at an extremely high rate, compared to the expectation from gnomad (>1000 fold increase), and are observed in 10 or more patients in the cohort. Network prioritization provides a link between observed MD SNPs and/or IN/DELs, candidate diagnostic MD genes, phenotype and molecular mechanisms for targeted therapies.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present disclosure provides a method for diagnosis and treatment of Ménière's disease (MD). In certain embodiments, the present disclosure provides that certain SNPs and/or IN/DELs in certain genes are responsible for a large proportion of the genetic load for Meniere's disease (MD). DNA samples from 527 patients were collected and 30X whole genome sequencing was performed. Network prioritization provides a link between observed MD SNPs and/orIN/DELs, candidate diagnostic MD genes, phenotype and molecular mechanisms for targeted therapies and a MD diagnostic panel that includes SNPs and/or IN/DELs in genes, such as FAM136A, ANKRD36, ANKRD36C, RBFOX1, GXYLT1, FAM8A1, ATP2B2, AP3S1, DYNC1H1, FLNA, KIFAP3 and AARS1.

The present disclosure further provides a method of diagnosis of Ménière's disease (MD) by genetic testing of SNPs and/or IN/DELs in certain genes disclosed herein that associate with MD of a subject of interest.

The present disclosure further provides a method of treating Ménière's disease (MD) by targeting SNPs and/or IN/DELs in certain genes disclosed herein. In certain embodiments, the present disclosure provides a method of treating Ménière's disease (MD) by administering to a subject of interest a therapeutically effective amount of a drug that targets the SNPs and/or IN/DELs, genes and/or molecular pathways disclosed herein. In certain embodiments, such drug includes, but is not limited to, Amelioride, which is a potassium sparing diuretic and is effective in the management of hypertension and sodium reabsorption in patients with genetic alteration of ANKRD36. Other known and/or to be known as members of class of pyrazinoylguanidine compounds can also be used for treating Meniere's disease (MD) by targeting ANKRD36 and/or ANKRD36C. Intratympanic administration is the prime method of administration, but other methods can be used.

In certain embodiments, the present disclosure provides gene therapy for the treatment of Meniere's disease (MD) using CRISPR-Cas9 or gene editing of SNP/INDELs in certain genes disclosed herein. For instance, for patients with Ménière's disease (MD) that possess a mutation in ANKRD36 and/or ANKRD36C gene that could be treated using CRISPR or gene therapy.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a catalyst,” “a metal,” or “a substrate,” includes, but are not limited to, mixtures or combinations of two or more such catalysts, metals, or substrates, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Unless otherwise specified, temperatures referred to herein are based on atmospheric pressure (i.e., one atmosphere).

The present disclosure presents the first whole-genome sequence analysis for genetic variation associated with well-characterized classical MD consisting of attacks of fluctuating unilateral hearing loss (low frequency), roaring tinnitus, and vertigo. Network prioritization provides a link between observed MD SNPs and/orIN/DELs, candidate diagnostic MD genes, phenotype and molecular mechanisms for targeted therapies.

Whole genome sequencing was performed on a cohort of 527 samples. SNPs and/or IN/DELs were called using bcbio¹ which implemented GATK 4.1.9 best practices pipeline for joint genotyping on the hg38 reference (Chapman et al., bcbio/bcbio-nextgen: Zenodo, 2021; doi: 10.5281/ZENODO.3564938). To call copy number variants, CNVkit (Talevich et al., PLOS Comput. Biol. 12, e1004873, 2016). was used and significantly amplified and deleted regions were identified using GISTIC (Mermel et al., Genome Biol. 12, R41, 2011). All samples passed quality control checks and were included in the analysis. Variants were filtered by gnomad v3.1.2, ExAC, 1000 genomes, to exclude variants common in >5% of the population. Variants deemed “benign” by Sift and Polyphen have been removed. Only protein altering variants were included. A local FDR was computed based on the observed frequency in the population, and the expected frequency from gnomad (v3 popmax). Variants with Ifdr<0.1 were kept. Following this filter 53,480 unique variants, including 8,448 which recurred (were found in more than one patient) were retained.

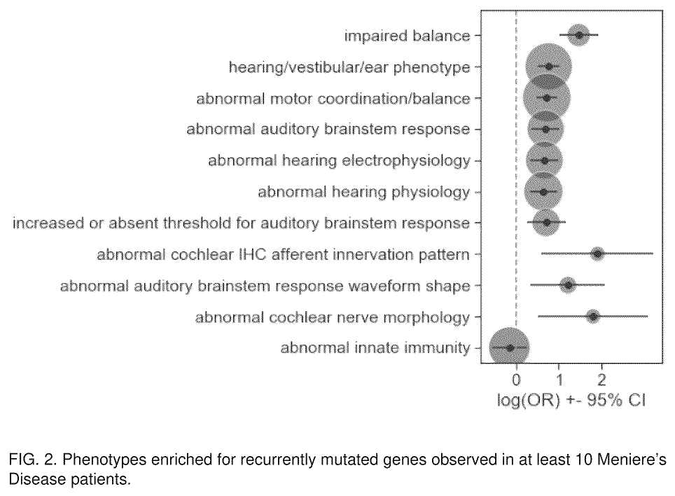

All genes mutated in more than 10 patients were integrated with a public database connecting mouse genotypes to phenotypes. There are 816 such genes in these data. A significant enrichment was found between these 816 genes and phenotypes related to abnormal hearing, auditory brainstem response, abnormal cochlear morphology, and impaired balance (FIG. 2). Perturbations in ATP2B2 are known to impact balance, hearing, and cochlear morphology. Other genes connected to abnormal hearing include CACNA1D, NIPBL, SPTBN4. Genes connected to balance include KCNC3, SOD2, ECC6 (FIG. 2). Such an overlap between genes found recurrently mutated in MD and those same genes impacting relevant phenotypes in mouse when mutated provides evidence that genes and variants identified in the study drive MD.

Gene set enrichment analysis (GSEA) was used to further prioritize genes in pathways relevant to MD. For example, RBFOX1 is involved in neuromuscular process controlling balance (GO:0050885), and ATP2B2 has a role in sensory perception of sound (GO:0007605). The Cellular Component (CC) gene ontology offers a different point of view on the etiology of MD. GSEA analysis of CC sets curated by MSigDB leads to 98 gene sets with positive enrichment score and high significance (Ifdr<0.01). Of those, plasma membrane-bound cell projection cytoplasm (GO:0032838), cell cortex (GO:0005938), adherens junction (GO:0005912), and actin-based cell projection (GO:98858) together explain 93.0% of cases. These make sense for MD from the phenomenological point of view: plasma membrane-bound cell projection cytoplasm combined with actin-based cell projection contain structural components of stereocilia and surrounding cytoplasmic proteins, which are the primary physical components of sound wave detection and the first step of sensory mechanotransduction. Adherens junctions are contact regions between epithelial cells, which normally maintain proper ion concentrations on both sides of the epithelial layer and by extension maintain osmotic balance across the membrane. Finally, cell cortex is a specialized layer of actin and other cytoplasmic proteins adjacent to the cell membrane that is responsible for maintaining cell shape and provides tissue integrity when many cells' cortexes are linked together via adherens junctions. Stereocilia are also physically rooted in the cell cortex. Cell cortex defects could manifest themselves as epithelial barrier leakage as well as impaired auditory and vestibular sensing. Cell cortex (GO:0005938) by itself explains 75.9% of patient cases. Taken together, these seven GO sets define a union gene set, which explains 99.0% of cases. Surprisingly, this relatively small set of functionally related biological processes and cellular components explains almost all cases. In terms of genes, these seven GO sets still define a relatively large number of genes (720). However, when sorting these pathway genes by network z-score, the top 7 genes, RBFOX1, ATB2B2, AP3S1, DYNC1H1, FLNA, KIFAP3, and AARS1, are found mutated in 80% of the cohort.

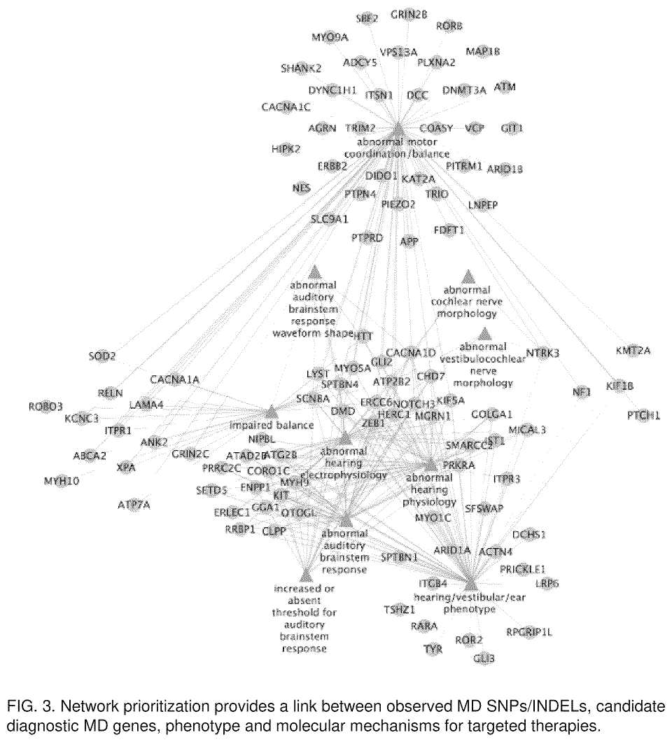

Furthermore, when compared the results for relevant phenotypes from the mouse variant database using the network reprioritized genes, the enrichment improves in key phenotypes as compared to the results ranked by pure mutation frequency (FIG. 2), including impaired motor coordination and balance, abnormal hearing physiology, and abnormal auditory brainstem response (FIG. 3). This result provides further support that the network-prioitized gene list provides better candidates for MD genes.

This is the first whole-genome sequence analysis of over 500 patients with very well characterized unilateral Ménière's disease (MD). The present disclosure presents the first to define the genetic architecture of this polygenic disease, and the first of its kind, and provides the bases for a deeper dive into the biology of MD and possible therapeutic targets.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Whole genome sequencing. All samples were initially purified using Ampure XP beads (0.8:1 sample to bead ratio). Genomic DNA quality was assessed using Genomic DNA Screen Tape on an Agilent 4200 (Agilent Technologies, Santa Clara, CA, USA), and quantity using the Qubit dsDNA HS (High Sensitivity) assay. Samples with DNA Integrity Number (DIN) greater than 4.0 and at least 500 nanogram (ng) of DNA were selected for subsequent processing. 500 ng of Genomic DNA from each sample was fragmented by Adaptive Focused Acoustics (E220 Focused Ultrasonicator, Covaris, Woburn, Massachusetts) to produce an average fragment size of 400 basepairs (bp). Sequencing libraries were generating using the KAPA Hyper Prep Kit (KAPA Biosystems, Wilmington, MA, USA) following manufacturer's instructions using 3 cycles of amplification. The quality of the library was assessed using High Sensitivity D1000 kit on a 4200 TapeStation instrument. Sequencing was performed using the NovaSeq 6000 Sequencing System (Illumina, San Diego, CA, USA), generating 150 bp paired-end reads to obtain 30X average coverage.

### Variant Calling

Whole genome sequencing was performed on a cohort of 527 samples. SNPs and/or IN/DELs were called using bcbio¹ which implemented GATK 4.1.9 best practices pipeline for joint genotyping on the hg38 reference (Chapman et al., bcbio/bcbio-nextgen: Zenodo, 2021; doi: 10.5281/ZENODO.3564938). To call copy number variants, CNVkit (Talevich et al., PLOS Comput. Biol. 12, e1004873, 2016) was used and then significantly amplified and deleted regions were identidied using GISTIC (Mermel et al., Genome Biol. 12, R41 (2011). All samples passed quality control checks and were included in the analysis.

### Identifying Recurrent Rare Variants

Variants were filtered by gnomad v3.1.2, ExAC, 1000 genomes, to exclude variants common in >5% of the population. Variants deemed “benign” by Sift and Polyphen have been removed. Onlyprotein altering variants were included. A local FDR was computed based on the observed frequency in the population, and the expected frequency from gnomad (v3 popmax). variants with Ifdr<0.1 were kept. Following this filter 53,480 unique variants, including 8,448 which recurred (were found in more than one patient) were retained.

### Network Analysis Methods

Tissue-specific gene interaction network for cochlea was obtained by filtering the generic STRING (Szklarczyk et al., Nucleic Acids Res. 43, D447-52, 2015) interaction network for genes expressed in the human cochlea. STRING version 11.5 was used, with all edges, in a weighted version of network propagation with weights of edges given by confidence. Expression data were obtained from RNASTAR. Mutations detected in the cohort of 527 Meniere's patients were used as seeds for the network propagation algorithm. Network propagation was used to score all genes. The calculated propagation score was compared to that of a null ensemble in which mutations in each patient are unrelated to MD. This is achieved in a rather primitive way by assuming that the mutations in the null ensemble occur with uniform probability in all genes. 10⁴ independent samples of 527 patients were generated, each from the null ensemble, with numbers of mutations the same as observed in the MD cohort but selected uniformly randomly in each patient. Genes are then sorted by their z-score. genes with large positive z were most interested in. The ranking of genes correlates with their mutation frequency but is not the same because z reflects connectivity among genes and so can amplify or dilute the effect of mutations. The collection of z-scores was used for Gene Set Enrichment Analysis (GSEA) with gene sets defined by the Gene Ontology (GO) consortium as Biological Process (BP), Molecular Function (MF) and Cellular Component (CC) supersets. The p-values of gene sets were converted to posterior error probabilities (Ifdr) using Storey's algorithm. Only gene sets with positive and significant GSEA scores (ES>0, Ifdr<0.01) are relevant. Positive GSEA scores indicate higher than expected network propagation signal in the patient cohort, and thus higher than expected mutation frequency of the leading edge genes themselves or that of their close network neighbors. On the other hand, significant gene sets with negative GSEA scores represent gene sets which are protected from random mutations.

### Example 2

### Population-Level Analyses

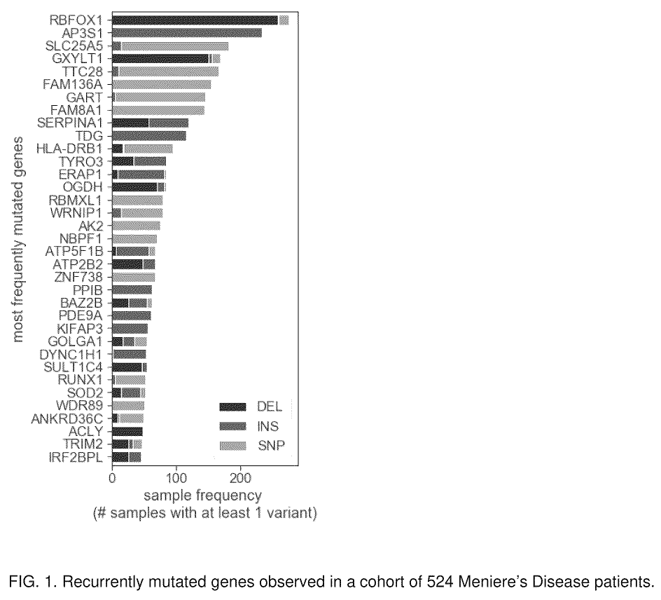

Notably, 281 variants were observed in 10 or more samples (FIG. 1). These include an in-frame deletion in RBFOX1 (p.Ser31del), found mutated in 258 patients (˜50%), an 18 fold increase over expectation, a missense mutation in FAM136A (p.Asp195His), mutated in 153 samples, a 9-fold increase over expectation, a nonsense mutation in GXYLT1 (p.Leu223_Arg224delinsTer), found in 151 samples, an 87 fold-increase over expectation, and a missense mutation in FAM8A1 (p.Leu135Pro), found in 99 samples, a 16-fold increase over expectation.

One of the most striking variants is a frameshift deletion in ATP2B2, a known variant (p.Thr927Ter; rs1438373024) so rare that it is not observed a single time in the gnomad V3 database, but is observed 48 times in these data. ATP2B2 encodes an ion channel pump that maintains homeostasis in hair cells and has been previously linked to hearing loss. Furthermore, numerous mouse models with alterations in Atp2b2 result in hearing deficits, by way of cochlear and vestibular hair cell abnormalities, among others. Along with the frameshift deletion mentioned above, 32 other unique variants were also found in ATP2B2 (passing the filtering criteria; FIG. 2. Most of these are long novel indels, observed only once or twice in the population. Altogether, 68 samples in these data have a variant in ATP2B2.

Other variants of interest include a nonsense mutation in ANKRD36(p.GIn481Ter), mutated 26 times in these data (a 57-fold increase over expectation). In ANKRD36C, both a nonsense mutation (p.Glu393Ter), found 33 times in the data (a 31-fold increase over expectation), and a frameshift deletion (p.Glu1279Ter), found 11 times in the data (an 889-fold increase over expectation) were observed.

### Example 3

Replication with Common Variants

A recent genome-wide association study (GWAS) of vertigo from a large cohort of individuals of European ancestry uncovered six significant loci, mapped to 6 genes (Skuladottir et al., Commun Biol 2021;4(1):1148). Of these six genes, ZNF91, OTOGL, TECTA, and ARMC9 had rare variants in the cohort.

Another large GWAS on motion sickness identified 35 significantly associated loci and identified genes associated with these loci which are known to be involved in balance, inner ear development, and other neurological processes (Bethann et al., Human Molecular Genetics, Volume 24, Issue 9, 1 May 2015, Pages 2700-2708). Of the 35 candidate genes associated with the motion sickness loci, 16 also had rare variants in the data (TSHZ1, ARAP2, ACO1, NR2F2, CNTN1, CELF2, AGA, TLE4, GXYLT2, TUSC1, NLGN1, AUTS2, UBE2E2, MAP2K5, and RGS5). Interestingly, GXYLT2 was identified as a candidate gene for motion sickness. While 3 rare variants were observed in this gene in this study, it was noted that a closely interacting gene, GXYLT1, is one of the most frequently mutated genes (FIG. 1), with the most recurrent variant, a nonsense deletion (rs1195046337), occurring 151 times in the cohort, 87 times more frequently than expected based on the gnomad population frequencies.

These results point to a possible overlap in mechanism between MD, vertigo, and motion sickness.

### Example 4

Replication with Mouse Variant Database

All genes mutated in more than 10 patients were integrated with a public database connecting mouse genotypes to phenotypes. There are 816 such genes in these data. A significant enrichment was found between these 816 genes and phenotypes related to abnormal hearing, auditory brainstem response, abnormal cochlear morphology, and impaired balance (FIG. 2). Perturbations in ATP2B2 are known to impact balance, hearing, and cochlear morphology. Other genes connected to abnormal hearing include CACNA1D, NIPBL, SPTBN4. Genes connected to balance include KCNC3, SOD2, ECC6 (FIG. 2). Such an overlap between genes found recurrently mutated in MD and those same genes impacting relevant phenotypes in mouse when mutated provides evidence that genes and variants identified MD.

### Example 5

### Network Analysis

By leveraging prior biological knowledge in the form of molecular interaction networks, biological pathways, and network propagation, the ‘universal amplifier of genetic associations’⁴, the list of genes and variants was reprioritized in the MD cohort. The reprioritized list boosts signal from real variants, while downweighting false positives, since false positive hits are less likely to interact in the network. 77 genes were identified with z>7 (FIG. 3). Of these reprioritized genes, ATP2B2, ACAT2, AK2, PC, and MAT2A have a variant mutated at an extremely high rate, compared to the expectation from gnomad (>1000 fold increase), and were observed in 10 or more patients in the cohort.

GSEA was used to further prioritize genes in pathways relevant to MD. For example, RBFOX1 is involved in neuromuscular process controlling balance (GO:0050885), and ATP2B2 has a role in sensory perception of sound (GO:0007605). The Cellular Component (CC) gene ontology offers a different point of view on the etiology of MD. GSEA analysis of CC sets curated by MSigDB leads to 98 gene sets with positive enrichment score and high significance (Ifdr<0.01). Of those, plasma membrane-bound cell projection cytoplasm (GO:0032838), cell cortex (GO:0005938), adherens junction (GO:0005912), and actin-based cell projection (GO:98858) together explain 93.0% of cases. These make sense for MD from the phenomenological point of view: plasma membrane-bound cell projection cytoplasm combined with actin-based cell projection contain structural components of stereocilia and surrounding cytoplasmic proteins, which are the primary physical components of sound wave detection and the first step of sensory mechanotransduction. Adherens junctions are contact regions between epithelial cells, which normally maintain proper ion concentrations on both sides of the epithelial layer and by extension maintain osmotic balance across the membrane. Finally, cell cortex is a specialized layer of actin and other cytoplasmic proteins adjacent to the cell membrane that is responsible for maintaining cell shape and provides tissue integrity when many cells' cortexes are linked together via adherens junctions. Stereocilia are also physically rooted in the cell cortex. Cell cortex defects could manifest themselves as epithelial barrier leakage as well as impaired auditory and vestibular sensing. Cell cortex (GO:0005938) by itself explains 75.9% of patient cases. Taken together, these seven GO sets define a union gene set, which explains 99.0% of cases. Surprisingly, this relatively small set of functionally related biological processes and cellular components explains almost all cases. In terms of genes, these seven GO sets still define a relatively large number of genes (720). However, when sorting these pathway genes by network z-score, the top 7 genes, RBFOX1, ATB2B2, AP3S1, DYNC1H1, FLNA, KIFAP3, and AARS1, are found mutated in 80% of the cohort (FIG. 3).

Furthermore, when compared the results for relevant phenotypes from the mouse variant database using the network reprioritized genes, the enrichment improves in key phenotypes as compared to the results ranked by pure mutation frequency (FIG. 2), including impaired motor coordination and balance, abnormal hearing physiology, and abnormal auditory brainstem response (FIG. 3). This result provides further support that the network-prioritized gene list provides better candidates for MD genes.

### REFERENCES

Bethann et al., Genetic variants associated with motion sickness point to roles for inner ear development, neurological processes and glucose homeostasis, Human Molecular Genetics, Volume 24, Issue 9, 1 May 2015, Pages 2700-2708.
Cabrera et al., Intronic variants in the NFKB1 gene may influence hearing forecast in patients with unilateral sensorineural hearing loss in meniere's disease. PLoS One 9, e112171 (2014).
Campbell et al., Polymorphisms in KCNE1 or KCNE3 are not associated with Ménière disease in the Caucasian population. Am. J. Med. Genet. A 152A, 67-74 (2010).
Candreia et al., 2010 PMID:21063116.
Chapman et al., bcbio/bcbio-nextgen:Zenodo, 2021), doi:10.5281/ZENODO.3564938.
Cohen & Jenkin, 1995, PMID: 7803019.
Doi et al., Ménière's disease is associated with single nucleotide polymorphisms in the human potassium channel genes, KCNE1 and KCNE3. ORL J. Otorhinolaryngol. Relat. Spec. 67, 289-293 (2005).
Furuta et al., Association of interleukin-1 gene polymorphisms with sudden sensorineural hearing loss and Ménière's disease. Int. J. Immunogenet. 38, 249-254 (2011).
Gabrikova et al., 2010 PMID:20927121.
Gázquez et al., Functional variants of MIF, INFG and TFNA genes are not associated with disease susceptibility or hearing loss progression in patients with Ménière's disease. Eur. Arch. Otorhinolaryngol. 270, 1521-1529 (2013).

Hietikko et al., A replication study on proposed candidate genes in Ménière's disease, and a review of the current status of genetic studies. Int. J. Audiol. 51, 841-845 (2012).
Jen, 2011 PMID: 21358195.
Klar et al., 2006 PMID: 16741942.
Li et al., Variants in the KCNE1 or KCNE3 gene and risk of Ménière's disease: A meta-analysis. J. Vestib. Res. 25, 211-218 (2016).
Lopes et al., Ménière's Disease: Molecular Analysis of Aquaporins 2, 3 and Potassium Channel KCNE1 Genes in Brazilian Patients. Otol. Neurotol. 37, 1117-1121 (2016).
Lopez-Escamez et al., 2009, PMID: 19663372.
Maekawa et al., 2010 PMID:20722976.
Mermel et al., GISTIC2.0 facilitates sensitive and confident localization of the targets of focal somatic copy-number alteration in human cancers. Genome Biol. 12, R41 (2011).
Morris et al., 1994, PMID: 8109627.
Morris et al., 2009 PMID:18616841.

Nishio, N. et al. Polymorphisms in genes encoding aquaporins 4 and 5 and estrogen receptor α in patients with Ménière's disease and sudden sensorineural hearing loss. Life Sci. 92, 541-546 (2013).
Ohmen et al., 2013 PMID: 2359870.
Skuladottir et al., A genome-wide meta-analysis uncovers six sequence variants conferring risk of vertigo. Commun Biol 2021;4(1):1148.
Szklarczyk et al., STRING v10: protein-protein interaction networks, integrated over the tree of life. Nucleic Acids Res. 43, D447-52 (2015).
Talevich et al., CNVkit: Genome-Wide Copy Number Detection and Visualization from Targeted DNA Sequencing. PLoS Comput. Biol. 12, e1004873 (2016).
Teggi et al., 2008 PMID: 18667944.

1. A method of diagnosing a patient with Ménière's disease (MD) comprising performing a genetic testing using a panel of Ménière's disease-associated gene mutation in one or more gene, and providing a genetic basis for MD.

2. The method of claim 1, wherein the gene providing genetic basis for MD comprises a gene selected from the group consisting of FAM136A, ANKRD36, ANKRD36C, RBFOX1, GXYLT1, FAM8A1, ATP2B2, AP3S1, DYNC1H1, FLNA, KIFAP3 and AARS1.

3. The method of claim 1, wherein the gene mutation comprises single nucleotide polymorphisms (SNPs), insertions and/or deletions (IN/DELs).

4. A method of treating Ménière's disease (MD) of a patient comprising administering to the patient with Ménière's disease (MD) an effective amount of an agent, wherein said agent targets one or more gene mutation in one or more Ménière's disease-associated gene.

5. The method of claim 4, wherein said Ménière's disease-associated gene comprises a gene selected from the group consisting of FAM136A, ANKRD36, ANKRD36C, RBFOX1, GXYLT1, FAM8A1, ATP2B2, AP3S1, DYNC1H1, FLNA, KIFAP3 and AARS1.

6. The method of claim 4, wherein the gene mutation comprises single nucleotide polymorphisms (SNPs), insertions and/or deletions (IN/DELs).

7. The method of claim 4, wherein said Ménière's disease-associated gene is ANKRD36 and/or ANKRD36C gene.

8. The method of claim 7, wherein said targeted agent is Amiloride.

9. The method of claim 8, wherein Amiloride is intratympanic administered.

10. A method of treating Ménière's disease (MD) of a patient comprising performing CRISPR or gene therapy or editing on one or more gene mutation in one or more Ménière's disease-associated gene of said patient.

11. The method of claim 10, wherein said Ménière's disease-associated gene comprises a gene selected from the group consisting of FAM136A, ANKRD36, ANKRD36C, RBFOX1, GXYLT1, FAM8A1, ATP2B2, AP3S1, DYNC1H1, FLNA, KIFAP3 and AARS1.

12. The method of claim 10, wherein the gene mutation comprises single nucleotide polymorphisms (SNPs), insertions and/or deletions (IN/DELs).
